(19) United States
(12) Patent Application Publication
(10) Pub. No.: US 2022/0192508 A1
(43) Pub. Date: Jun. 23, 2022

(54) WRISTBAND PULSE DIAGNOSIS INSTRUMENT

(71) Applicant: NATIONAL DONG HWA UNIVERSITY, Hualien (TW)

(72) Inventors: SHI-JIM YEN, Hualien County (TW); WEN-CHIH CHEN, Hualien County (TW); XIAN-DONG CHIU, Hualien County (TW); YU-JIN LIN, Taipei City (TW)

(21) Appl. No.: 17/126,360

(22) Filed: Dec. 18, 2020

(51) Int. Cl. A61B 5/022; A61B 5/00

(52) U.S. Cl. CPC A61B 5/022; A61B 2562/227; A61B 2562/0247; A61B 5/681

(57) ABSTRACT

A wristband pulse diagnosis instrument including two pulse diagnosis instrument units is provided. Each of the two pulse diagnosis instrument units includes a wristband body, a pressure sensor, a first adjusting mechanism, and a second adjusting mechanism. A connecting member is disposed at each of two sides of the wristband body, and the connecting members are used to selectively connect the two pulse diagnosis instrument units to each other. The pressure sensor is movably disposed on the wristband body via the first adjusting mechanism and the second adjusting mechanism. A depthwise movement of the pressure sensor is adjusted by the first adjusting mechanism for the pressure sensor to move to three pulse positions of floating, medium, and deep, and a lateral movement of the pressure sensor is adjusted by the second adjusting mechanism for the pressure sensor to move to three pulse positions of distal, mid, and proximal.

### FIELD OF THE DISCLOSURE

The present disclosure relates to a pulse diagnosis instrument, and more particularly to a wristband pulse diagnosis instrument for pulse diagnosis.

### BACKGROUND OF THE DISCLOSURE

Pulse diagnosis is an important diagnosis method in the field of Chinese medicine for diagnosing diseases and monitoring effect of treatments. Doctors practicing traditional Chinese medicine take pulses (i.e., performing pulse diagnosis) by placing three fingers (mainly the index finger, the middle finger, and the ring finger) upon the radial artery of a wrist of a patient, and sense a reaction of the veins of the patient under different degrees of pressure exerted by the three fingers, so as to perform a pulse analysis. From the perspective of modern medicine, the theoretical basis and clinical value of the pulse diagnosis are still questionable. However, from the perspective of Chinese medicine, diagnostic information obtained from the pulse diagnosis is beyond doubt. In the past, doctors of traditional Chinese medicine relied on accumulation of experience and knowledge to perform pulse diagnosis by hand. However, the sensitivity of one doctor can be different from that of another doctor, and passing down the experience can be difficult. Although some pulse diagnosis instruments are already in use, most of the conventional pulse diagnosis instruments are large in size, and are not portable and not convenient to be worn. Furthermore, the conventional pulse diagnosis instruments can only measure a single pulse position, and are incapable of measuring three pulse positions of floating (Fu), medium (Zhong), and deep (Chen), and three pulse positions of distal (Cun), mid (Guan), and proximal (Chi), or measuring pulses of both hands simultaneously.

### SUMMARY OF THE DISCLOSURE

In response to the above-referenced technical inadequacies, the present disclosure provides a wristband pulse diagnosis instrument that is portable and convenient to be worn, can measure three pulse positions of floating, medium, and deep and three pulse positions of distal, mid, and proximal, and can also measure pulses of both hands simultaneously.

In one aspect, the present disclosure provides a wristband pulse diagnosis instrument including two pulse diagnosis instrument units. Each of the two pulse diagnosis instrument units includes a wristband body, a pressure sensor, a first adjusting mechanism, and a second adjusting mechanism. The wristband body has an inner edge and an outer edge, the inner edge and the outer edge are respectively positioned on two sides of the wristband body opposite to each other, and the inner edge has an arced shape. A connecting member is disposed at each of the two sides of the wristband body, and the connecting members are used to selectively connect the two pulse diagnosis instrument units to each other, such that the two pulse diagnosis instrument units are used cooperatively with or separately from each other. The pressure sensor is movably disposed on the wristband body via the first adjusting mechanism and the second adjusting mechanism. A depthwise movement of the pressure sensor is adjusted by the first adjusting mechanism for the pressure sensor to move to the three pulse positions of floating, medium, and deep, and a lateral movement of the pressure sensor is adjusted by the second adjusting mechanism for the pressure sensor to move to the three pulse positions of distal, mid, and proximal.

Preferably, the first adjusting mechanism is a radial adjusting mechanism, and a movement adjustment of the pressure sensor is controlled by the first adjusting mechanism along a radial direction of the wristband body, such that the depthwise movement of the pressure sensor is adjusted.

Preferably, the second adjusting mechanism is an axial adjusting mechanism, and a movement adjustment of the pressure sensor is controlled by the second adjusting mechanism along an axial direction of the wristband body, such that the lateral movement of the pressure sensor is adjusted.

One of the beneficial effects of the wristband pulse diagnosis instrument provided in the present disclosure is that the wristband pulse diagnosis instrument includes two pulse diagnosis instrument units, and each of the two pulse diagnosis instrument units includes a wristband body, a pressure sensor, a first adjusting mechanism, and a second adjusting mechanism. The depthwise movement of the pressure sensor is adjusted by the first adjusting mechanism for the pressure sensor to move to the three pulse positions of floating, medium, and deep, and the lateral movement of the pressure sensor is adjusted by the second adjusting mechanism for the pressure sensor to move to the three pulse positions of distal, mid, and proximal. Therefore, the wristband pulse diagnosis instrument of the present disclosure is portable and convenient to wear, and the wristband pulse diagnosis instrument can measure the three pulse positions of floating, medium, and deep, and the three pulse positions of distal, mid, and proximal. Furthermore, the wristband pulse diagnosis instrument can measure the pulses of both hands simultaneously.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a perspective schematic view of a wristband pulse diagnosis instrument of the present disclosure.

FIG. 2 is a planar schematic view of the wristband pulse diagnosis instrument of the present disclosure.

FIG. 3 is a schematic view showing an adjusting state of the wristband pulse diagnosis instrument of the present disclosure.

FIG. 4 is a schematic view showing another adjusting state of the wristband pulse diagnosis instrument of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### EMBODIMENT

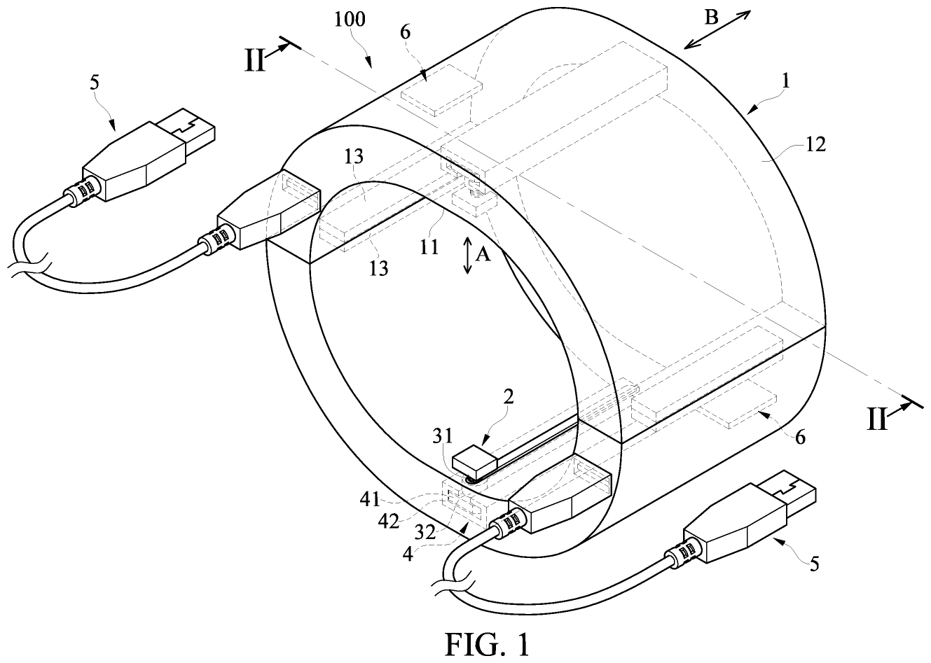

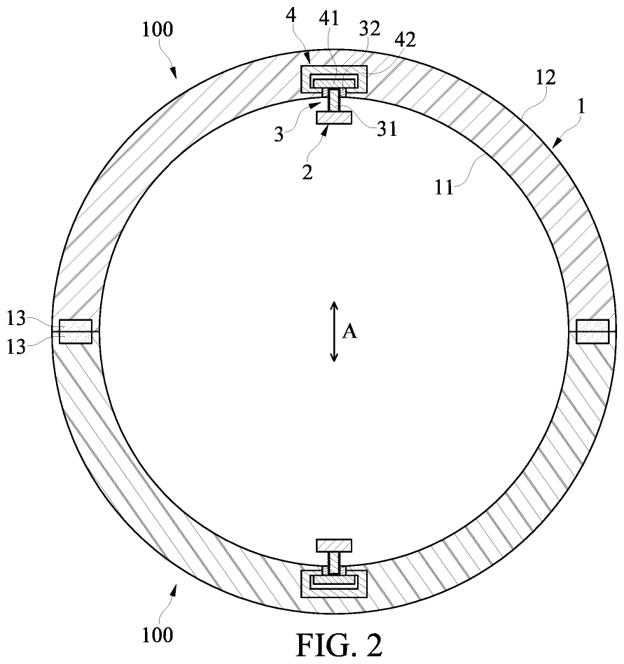

Referring to FIG. 1 and FIG. 2, a wristband pulse diagnosis instrument including two pulse diagnosis instrument units 100 is provided in the present disclosure. Each of the two pulse diagnosis instrument units 100 includes a wristband body 1, a pressure sensor 2, a first adjusting mechanism 3 and a second adjusting mechanism 4.

The wristband body 1 can be made of plastic materials, but the present disclosure is not limited thereto. For example, the wristband body 1 can be hollow and semi-circular, such that the wristband body 1 of the two pulse diagnosis units 100 can be combined to form a ring and can be worn on a wrist of a user. The wristband body 1 has an inner edge 11 and an outer edge 12. The inner edge 11 and the outer edge 12 are respectively positioned on two sides of the wristband body 1 opposite to each other. The inner edge 11 is curved, that is, the inner edge 11 can be in a shape of an arc. When the wristband body 1 is worn on the wrist of the user, the inner edge 11 of the wristband body 1 can face and be tightly attached to the wrist of the user.

A connecting member 13, which can be a magnetic structure or a buckle structure, etc., is disposed at each of the two sides of the wristband body 1, so as to connect the wristband body 1 of each of the two pulse diagnosis instrument units 100 to each other by way of magnetic attraction or buckling. Therefore, the two wristband bodies 1 can form a ring shape, and the two wristband bodies 1 can be separated easily, such that the two pulse diagnosis instrument units 100 can be used cooperatively with or separately from each other. In this way, the wristband pulse diagnosis instrument of the present disclosure can also measure pulses of both hands simultaneously. In this embodiment, the connecting member 13 is a magnetic structure, and the connecting member 13 can include a magnet or iron, so as to connect the wristband body 1 of each of the two pulse diagnosis instrument units 100 to each other by the magnetic attraction.

The pressure sensor 2 is a sensor that measures changes of pressure, and there is no limitation on the type of the pressure sensor 2 in the present disclosure. The pressure sensor 2 is movably disposed on the wristband body 1 via the first adjusting mechanism 3 and the second adjusting mechanism 4. The pressure sensor 2 is adjacent to a middle position of the wristband body 1, and a depthwise movement and a lateral movement of the pressure sensor 2 can be adjusted to accurately measure the pulse.

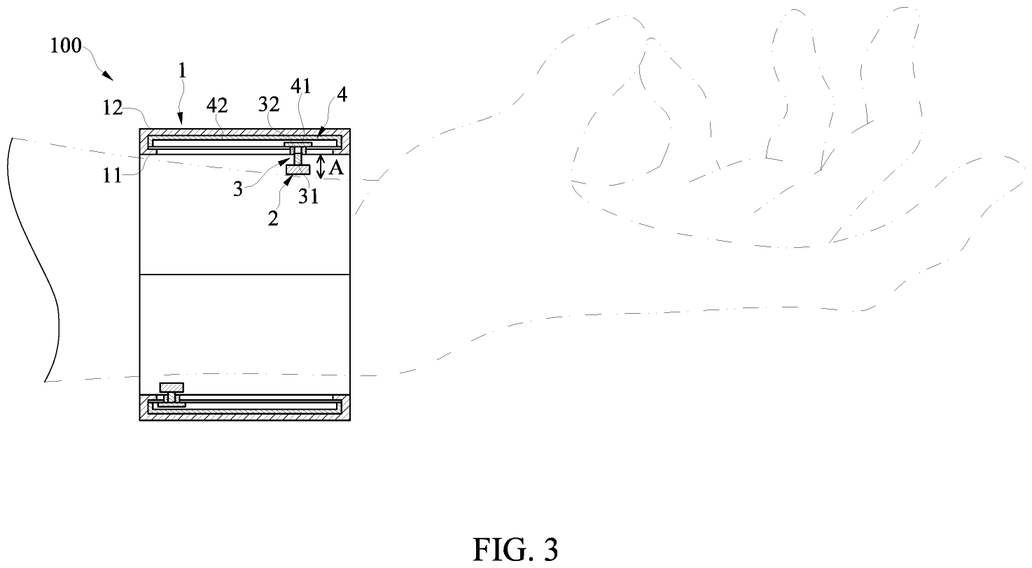

The first adjusting mechanism 3 is a radial adjusting mechanism. The first adjusting mechanism 3 can be adjusted manually, so as to move and adjust the pressure sensor 2 along a radial direction A of the wristband body 1, such that the pressure sensor 2 can be adjusted by moving depthwise. In this embodiment, the first adjusting mechanism 3 includes a screw 31 and a nut 32. The screw 31 and the nut 32 are respectively connected to the pressure sensor 2 and the wristband body 1, and the screw 31 and the nut 32 are threadedly engaged with each other. Therefore, the pressure sensor 2 can be moved and adjusted along the radial direction A through the threaded engagement between the screw 31 and the nut 32 (as shown in FIG. 3), such that a depth by which the pressure sensor 2 protrudes from the inner edge 11 of the wristband body 1 can be adjusted. In this embodiment, the depthwise movement of the pressure sensor 2 is designed by utilizing a threaded mechanism, so that the pressure sensor 2 can slowly move to three pulse positions of floating (Fu), medium (Zhong), and deep (Chen) of Chinese medicine.

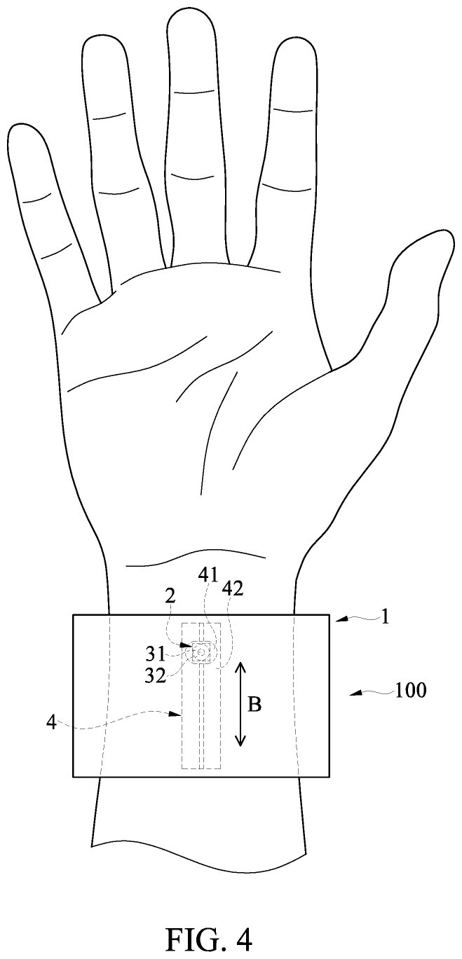

The second adjusting mechanism 4 is an axial adjusting mechanism, and the second adjusting mechanism 4 can be adjusted manually so as to move and adjust the pressure sensor 2 along an axial direction B of the wristband body 1 (as shown in FIG. 4). That is, the pressure sensor 2 can be controlled by the second adjusting mechanism 4 to move in a width direction of the wristband body 1, such that the pressure sensor 2 can be positioned on different positions along the axial direction B of the wristband body 1, and the pressure sensor 2 can be moved laterally. In this embodiment, the second adjusting mechanism 4 includes a slide block 41 and a slide rail 42, the slide block 41 and the slide rail 42 are respectively connected to the pressure sensor 2 and the wristband body 1, and the slide block 41 and the slide rail 42 are slidably engaged with each other. The slide block 41 can be connected to the pressure sensor 2 through the first adjusting mechanism 3, so that the pressure sensor 2 can be adjusted along the axial direction B through a sliding mechanism of the slide block 41 and the slide rail 42. In this embodiment, the pressure sensor 2 moves laterally through the sliding mechanism to correspond to the three pulse positions of floating (Fu), medium (Zhong), and deep (Chen) in Chinese medicine. There are three portions and nine indicators (San Bu Jiu Hou) in traditional Chinese medicine, in which the three portions refer to the three pulse positions of floating (Fu), medium (Zhong), and deep (Chen), and the nine indicators refer to the three pulse positions of distal (Cun), mid (Guan), and proximal (Chi). The above designs can also be measured manually by doctors practicing Chinese medicine.

In this embodiment, each of the two pulse diagnosis instrument units 100 further includes an electrical connector 5 which is electrically connected to the pressure sensor 2, and a pulse diagnosis main board 6 can be disposed in the wristband body 1. The pulse diagnosis main board 6 can be a circuit board. The pulse diagnosis main board 6 is electrically connected to the pressure sensor 2 and the electrical connector 5. The pulse diagnosis main board 6 can be used to read a pressure value captured by the pressure sensor 2, and the pressure value is continuously transmitted to a personal computer (PC) end through the electrical connector 5. In addition, the pressure value captured by the pressure sensor 2 and read by the pulse diagnosis main board 6 can also be transmitted to the PC end through wireless transmission.

### Beneficial Effects of Embodiments

One of the beneficial effects of the wristband pulse diagnosis instrument provided in the present disclosure is that the wristband pulse diagnosis instrument includes two pulse diagnosis instrument units, and each of the two pulse diagnosis instrument units includes a wristband body, a pressure sensor, a first adjusting mechanism, and a second adjusting mechanism. The depthwise movement of the pressure sensor is adjusted by the first adjusting mechanism for the pressure sensor to move to the three pulse positions of floating, medium, and deep, and the lateral movement of the pressure sensor is adjusted by the second adjusting mechanism for the pressure sensor to move to the three pulse positions of distal, mid, and proximal. Therefore, the wristband pulse diagnosis instrument of the present disclosure is portable and convenient to wear, and the wristband pulse diagnosis instrument can measure the three pulse positions of floating, medium, and deep, and the three pulse positions of distal, mid, and proximal. Furthermore, the wristband pulse diagnosis instrument can measure the pulses of both hands simultaneously.

The wristband pulse diagnosis instrument of the present disclosure is provided through a design that combines medical expertise with wearable devices and digital signal processing technology. The wristband pulse diagnosis instrument greatly improves sensitivity and convenience of pulse diagnosis and repeatability in measurement, which is beneficial to clinical application of Chinese medicine.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A wristband pulse diagnosis instrument, the wristband pulse diagnosis instrument including two pulse diagnosis instrument units, each of the two pulse diagnosis instrument units comprising:
a wristband body having an inner edge and an outer edge, the inner edge and the outer edge being respectively positioned on two sides of the wristband body opposite to each other, and the inner edge having an arced shape, wherein a connecting member is disposed at each of the two sides of the wristband body, and the connecting members are used to selectively connect the two pulse diagnosis instrument units to each other, such that the two pulse diagnosis instrument units are used cooperatively with or separately from each other;
a pressure sensor;
a first adjusting mechanism; and
a second adjusting mechanism;
wherein the pressure sensor is movably disposed on the wristband body via the first adjusting mechanism and the second adjusting mechanism;
wherein a depthwise movement of the pressure sensor is adjusted by the first adjusting mechanism for the pressure sensor to move to three pulse positions of floating, medium, and deep, and a lateral movement of the pressure sensor is adjusted by the second adjusting mechanism for the pressure sensor to move to three pulse positions of distal, mid, and proximal.

2. The wristband pulse diagnosis instrument according to claim 1, wherein the first adjusting mechanism is a radial adjusting mechanism, and a movement adjustment of the pressure sensor is controlled by the first adjusting mechanism along a radial direction of the wristband body, such that the depthwise movement of the pressure sensor is adjusted.

3. The wristband pulse diagnosis instrument according to claim 2, wherein the first adjusting mechanism includes a screw and a nut, the screw and the nut are connected to the pressure sensor and the wristband body, respectively, and the screw and the nut are threadedly engaged with each other.

4. The wristband pulse diagnosis instrument according to claim 1, wherein the second adjusting mechanism is an axial adjusting mechanism, and a movement adjustment of the pressure sensor is controlled by the second adjusting mechanism along an axial direction of the wristband body, such that the lateral movement of the pressure sensor is adjusted.

5. The wristband pulse diagnosis instrument according to claim 4, wherein the second adjusting mechanism includes a slide block and a slide rail; wherein the slide block and the slide rail are connected to the pressure sensor and the wristband body, respectively; wherein the slide block and the slide rail are slidably engaged with each other, such that the pressure sensor is adjusted along the axial direction.

6. The wristband pulse diagnosis instrument according to claim 1, wherein each of the two pulse diagnosis instrument units includes an electrical connector that is electrically connected to the pressure sensor.

7. The wristband pulse diagnosis instrument according to claim 6, wherein the wristband body further includes a pulse diagnosis main board, and the pulse diagnosis main board is electrically connected to the pressure sensor and the electrical connector.

8. The wristband pulse diagnosis instrument according to claim 1, wherein the wristband body of each of the two pulse diagnosis instrument units is hollow and semi-circular, and when the two pulse diagnosis instrument units are used cooperatively with each other, the two pulse diagnosis instrument units are assembled with each other to form a ring shape.

9. The wristband pulse diagnosis instrument according to claim 1, wherein the connecting members are magnetic structures or buckle structures.
